(19) United States
(12) Patent Application Publication
(10) Pub. No.: US 2005/0186136 A1
(43) Pub. Date: Aug. 25, 2005

(54) Soluble JAM-1, 2, and 3 as diagnostic & prognostic markers in cardiovascular, vascular, and inflammatory disorders

(76) Inventor: Shaker Mousa, Wynantskill, NY (US)

Correspondence Address: Vascular Vision Pharmaceuticals, 5 Fox Glove Ct., Wynantskill, NY, 12198, US

(21) Appl. No.: 10/783,453

(22) Filed: Feb. 23, 2004

(51) Int. Cl. A61K 51/00; G01N 33/53; A61K 39/395

(52) U.S. Cl. 424/1.49; 435/7.1; 424/143.1; 530/388.15

(57) ABSTRACT

This invention pertains to methods and composition of novel assays for the measurements of soluble JAM-1, 2, and 3 in body fluids for the diagnosis and therapeutic of various pathological disorders involved in cardiovascular, vascular, inflammatory, and angiogenesis-mediated disorders (cancer, ocular, inflammatory disorders). Additionally, these novel assays can be used to monitor therapeutics used for the prevention and treatment of these disorders.

### FIELD OF THE INVENTION

Junction adhesion molecules (JAMs) are an exciting new family of proteins that are located at intercellular junctions that might play a key role in leukocyte transmigration across the endothelium and hence inflammatory processes. Additionally, elevation of JAMs in body fluids might be used as a diagnostic and prognostic means of the degree and extent of various vascular, inflammatory, cardiovascular, hematological, ocular, oncological disorders. The JAM-1, 2 or 3 antibodies and fragments may be labeled and are useful in a variety of applications, such as in therapy in treating angiogenesis, inflammation-mediated conditions such as vascular, cardiovascular, cancer, rheumatoid arthritis, thrombosis, sickle cell disorder. Also provided is a kit for detecting the presence of human JAM-1, 2 or 3 antigens comprising an antibody or fragments of the invention, which is labeled for diagnostics and therapeutics in the above listed disorders. Measuring plasma, urine, tissue and cell extracts using kits for JAMs would provide a non-invasive method for the diagnosis of various disorders as well as in monitoring various therapeutic modalities for inflammatory, cardiovascular, and vascular disorders.

### BACKGROUND OF THE INVENTION

Cell adhesion is a complex process that is important for maintaining tissue integrity and generating physical and permeability barriers within the body. All tissues are divided into discrete compartments, each of which is composed of a specific cell type that adheres to similar or different cell types. Such adhesion triggers the formation of intercellular junctions (i.e., readily definable contact sites on the surfaces of adjacent cells that are adhering to one another), also known as tight junctions, gap junctions, spot and belt desmosomes. The formation of such junctions gives rise to physical and permeability barriers that restrict the free passage of cells and other biological substances from one tissue compartment to another. For example, the blood vessels of all tissues are composed of endothelial cells. In order for components in the blood to enter a given tissue compartment, they must first pass from the lumen of a blood vessel through the barrier formed by the endothelial cells of that vessel. Similarly, in order for substances to enter the body via the gut, the substances must first pass through a barrier formed by the epithelial cells of that tissue. To enter the blood via the skin, both epithelial and endothelial cell layers must be crossed.

Cell adhesion is mediated by specific cell surface adhesion molecules (CAMs). There are many different families of CAMs, including the immunoglobulin, integrin, selectin and cadherin superfamilies, and each cell type expresses a unique combination of these molecules. Cadherins are a rapidly expanding family of calcium-dependent CAMs (Munro et al., In: Cell Adhesion and Invasion in Cancer Metastasis, P. Brodt, ed., pp. 17-34, RG Landes Co., Austin Tex., 1996). The cadherins (CAD) are membrane glycoproteins that generally promote cell adhesion through homophilic interactions (a CAD on the surface of one cell binds to an identical CAD on the surface of another cell). Cadherins have been shown to regulate epithelial, endothelial, neural and cancer cell adhesion, with different CADs expressed on different cell types. For example, N (neural)-cadherin is predominantly expressed by neural cells, endothelial cells and a variety of cancer cell types. E (epithelial)-cadherin is predominantly expressed by epithelial cells. VE (vascular endothelial)-cadherin is predominantly expressed by endothelial cells. Other CADs are P (placental)-cadherin, which is found in human skin, and R (retinal)—cadherin. A detailed discussion of the cadherins is provided in Munro S B et al., 1996, In: Cell Adhesion and Invasion in Cancer Metastasis, P. Brodt, ed., pp. 17-34 (RG Landes Company, Austin Tex.) and Lampugnani and Dejana, Curr. Opin. Cell Biol. 9:674-682, 1997).

CAD-mediated cell adhesion triggers a cascade of events that lead to the formation of intercellular junctions, and ultimately to the establishment of permeability barriers between tissue compartments. The intercellular junction that is directly responsible for the creation of permeability barriers that prevent the diffusion of solutes through paracellular spaces is known as the tight junction, or zonula occludens (Anderson and van Itallie, Am. J. Physiol. 269:G467-G475, 1995; Lampugnani and Dejana, Curr. Opin. Cell Biol. 9:674-682, 1997).

The transmembrane component of tight junctions that has been the most studied is occludin (Furuse et al., J. Cell Biol. 123:1777-1788, 1993; Furuse et al., J. Cell Sci. 109:429-435, 1996). This protein appears to be expressed by all endothelial cell types, as well as by most epithelial cell types. Occludin is believed to be directly involved in cell adhesion and the formation of tight junctions (Furuse et al., J. Cell Sci. 109:429-435, 1996; Chen et al., J. Cell Biol 138:891-899, 1997). A detail of the occludin structure and function is provided by Lampugnani and Dejana, Curr. Opin. Cell Biol. 9:674-682, 1997.

More recently, junctional adhesion molecule (JAM) has been identified as an immunoglobulin gene superfamily member that is a component of tight junctions (Martin-Padura et al., J. Cell. Biol. 142:117-127, 1998). This protein is selectively concentrated at intercellular junctions of endothelial and epithelial cells of different origins, and has been shown to play a role in regulating monocyte transmigration.

Although cell adhesion is required for certain normal physiological functions, there are situations in which the level of cell adhesion is undesirable. For example, autoimmune diseases, graft rejection, and inflammatory diseases involve abnormal cellular adhesion.

In addition, permeability barriers arising from cell adhesion create difficulties for the delivery of drugs to specific tissues and tumors within the body. For example, skin patches are a convenient tool for administering drugs through the skin. However, the use of skin patches has been limited to small, hydrophobic molecules because of the epithelial and endothelial cell barriers. Similarly, endothelial cells render the blood capillaries largely impermeable to drugs, and the blood/brain barrier has hampered the targeting of drugs to the central nervous system. In addition, many solid tumors develop internal barriers that limit the delivery of anti-tumor drugs and antibodies to inner cells. Attempts to facilitate the passage of drugs across such barriers generally rely on specific receptors or carrier proteins that transport molecules across barriers in vivo. However, such methods are often inefficient, due to low endogenous transport rates or to the poor functioning of a carrier protein with drugs. While improved efficiency has been achieved using a variety of chemical agents that disrupt cell adhesion, such agents are typically associated with undesirable side effects, may require invasive procedures for administration and may result in irreversible effects.

### DETAILED DESCRIPTION OF THE INVENTION

JAM:

Cells are able to migrate into the inflamed tissue through a process involving additional cell adhesion molecules and chemokines. Junctional adhesion molecules (JAMs) are an exciting new family of proteins that are located at intercellular junctions. Available data provides evidence for their role in leukocyte transmigration across the endothelium. JAM-2 is homologous to the junctional adhesion molecule, JAM (JAM-1) and to VE-JAM (JAM-3). Several stretches of sequence conservation between JAM, JAM-2 and JAM-3 are also identified within their cytoplasmic domains. The relative tissue distribution of the transcript explored by northern blot indicates that it is prominently expressed in lymphoid organs, testis and kidney. Further analysis by immunohistochemistry indicates that the protein is highly expressed by high endothelial venules and lymphatic sinuses in lymphoid organs. The human gene is located on chromosome 11q25. When expressed in endothelial cells, all three molecules are localized at cell-cell contacts and co-localize with the ZO-1, a molecule found in junctional intercellular complexes. Thus, JAM-2, VE-JAM and JAM are the prototypes of a novel junctional adhesion molecule family. The JAM-2 molecule is part of the Ig supergene family and is expressed by a subpopulation of endothelial cells including HEV and is located in junctional structures of the vascular endothelium.

Upon partial cell-cell contact, the JAM-2 enriched membranes closed in a “zipper like” fashion, indicating that JAM-2 homophilic interaction is an early event in the establishment of cell connections and may play a role in cell-cell contact organization.

JAM-1 and JAM-2 not only have sequence similarities, but also have similar properties in term of sub-cellular localization, permeability regulation and homophilic interactions.

JAM Measurement: Principle of the Assay

Soluble JAM-1 assay utilizes an immuno-enzymometric technique for quantitation of JAM-1 in samples. This involves the simultaneous reaction of soluble JAM-1 present in the sample or standard to two antibodies directed against different epitopes on the soluble JAM-1 molecule. One antibody is coated onto the walls of the microtiter wells and the other is conjugated to the enzyme horseradish peroxidase (HRP). Any soluble JAM-1 present forms a bridge between the two antibodies. After removal of unbound material by aspiration and washing, the amount of conjugate bound to the well is detected by reaction with a substrate specific for the enzyme which yields a colored product proportional to the amount of conjugate (and thus soluble JAM-1 in the sample). The colored product can be quantified photometrically. By analyzing standards of known soluble JAM-1 concentration coincident with samples and plotting a curve of signal versus concentration, the concentration of unknowns can be determined.

The same principle is applied to JAM-2 and JAM-3.

Assay Procedures:

i. Dilute all samples at least 1 in 50 with sample diluents. For most samples (serum, plasma, or cell culture fluids) a dilution of 1 in 10 and 1 in 100 should be adequate.
ii. Use 96 microtiter plate
iii. Add 100 μL diluted Anti-JAM-1, 2 or 3-HRP Conjugate to each well.
iv. Add 100 μL Standard, diluted sample, or diluted Parameter Control to each well with sufficient force to ensure mixing. Shaking or tapping is not recommended.
v. Cover the plate with a plate sealer provided and incubate at room temperature for 1.5 hours.
vi. Aspirate or decant contents from each well and wash by adding 300 μl Wash Buffer per well. Repeat the process five times for a total of six washes.
vii. Immediately after decanting, add 100 μL Substrate to each well.
viii. Cover the plate with a plate sealer and incubate at room temperature for 30 minutes.
ix. Add 100 μL or Stop Solution to each well.
x. Determine the optical density (OD) of each well within 30 minutes using a microtiter plate reader or Photometer set at 450 nm with a correction wavelength of 620 nm. The photometer or plate reader should be blanked according to the manufacturer's instructions.
   JAM & Inflammatory Processes:

Inflammation is the body's natural defense mechanism against bacterial, viral and parasitic infections. The inflammatory cascade is a series of steps by which the body attempts to limit or destroy a foreign agent. One of the earliest events in the inflammatory cascade involves the binding of white blood cells to cell adhesion molecules called selectins that are expressed on the surface of blood vessel walls. This interaction between the leukocytes, white blood cells, and the selectins slows the flow of the white blood cells through the bloodstream and allows for the release of chemokines, small molecular weight cytokines involved in chemotaxis of both specific and overlapping subsets of leukocytes. Chemokines also activate a second class of cell adhesion molecules called integrins on the surface of the leukocytes. One such integrin, VLA-4, binds to vascular cell adhesion molecule-1 (VCAM-1) on the surface of the endothelial cells, and results in the firm attachment of the leukocytes to the vessel wall. At this point, the cells are able to migrate into the inflamed tissue through a process involving additional cell adhesion molecules and chemokines.

Junctional adhesion molecules (JAMs) are an exciting new family of proteins that are located at intercellular junctions. Available data provides evidence for their role in leukocyte transmigration across the endothelium and hence inflammatory processes.

JAM & Angiogenesis-Mediated Processes:

An Immunoglobulin similar to JAM is PECAM-1, an endothelial cell adhesion molecule whose expression is essential for endothelial cell-cell interactions during angiogenesis both in vitro and in vivo. In the past two years we have been investigating the role of PECAM-1 in angiogenesis.

These studies have involved identification of PECAM-1 isoforms expressed in different vascular beds in a developmentally regulated fashion, determination of the adhesive function of different isoforms in endothelial cells, identification of the signaling pathways activated by PECAM-1 isoforms, and determination of the interrelationship between PECAM-1 mediated and cadherin mediated cell-cell adhesion. PECAM-1 plays a role to be defined in angiogenesis/vasculogenesis. Similarly JAM-1 demonstrated key role in the modulation of angiogenesis by its cross-talk with ανβ3 integrin. This knowledge will be instrumental in understanding how angiogenesis is regulated and in the development of new agents inhibit it.

1. Diagnostic and prognostic assay kits for the measurement of JAM-1, 2, and 3 in body fluids including plasma, sera, urine, cell media, and tissue extracts. The kits can be used for detecting the presence of human JAM-1, 2 or 3 antigens comprising of an antibody or fragments, which is labeled for diagnostics and therapeutics.

2. The method of therapeutics wherein JAM-1, 2 or 3 antibodies and fragments may be used unlabelled or labeled in a variety of applications, such as in therapy for treating angiogenesis- and inflammation-mediated conditions such as vascular, ocular disorders, cardiovascular, cancer, rheumatoid arthritis, thrombosis, sickle cell disease.

3. The method of diagnosis wherein the label is selected from the group consisting of: (a) an enzyme label; (b) a radioisotope; (c) a fluorescent label; and (d) biotin.

4. The method of claim 3 wherein the label is a radioisotope selected from the group consisting a) I-125; (b) I-121; (c) I-131; (d) IN-111; and (e) Technetium (TC) 99 m.

5. A diagnostic kit of claim 3 wherein all reagents are together or individually to be used with 24, 48, 96 or any lager micro-titer well plates.

6. A labeled humanized antibody or fragment of claim 1, wherein said antibody or fragment is labeled with a label selected from the group consisting of a radionuclide, a fluor, an enzyme label, an enzyme substrate, enzyme cofactor, enzyme inhibitor and a hapten. 5.

7. A kit of claim 1 and 3 for detecting the presence of human JAM-1, 2 or 3 antigen, said kit comprising a humanized antibody or fragment wherein said antibody or fragment specifically binds to human JAM-1, 2 or 3.

8. The kit of claim 7, further comprising a second antibody reactive with said humanized antibody or fragment.

9. The kit of claim 7, wherein said second antibody is labeled with a label selected from the group consisting of a radionuclide, a fluor, an enzyme label, an enzyme substrate, enzyme cofactor, enzyme inhibitor and a hapten.

10. The method of claim 1-9 wherein the antibody or portion thereof is a monoclonal antibody.

11. The method of claim 1-9 wherein the antibody or portion thereof is a polyclonal antibody.

12. The method of claim 1-9 wherein the antibody or portion thereof is a Fab fragment.

13. The kit of claim 1 and 3, further comprising a second antibody reactive with said humanized antibody or fragment wherein said second antibody is labeled.

14. The method of claim 1 and 3 to be used in the diagnosis of various cardiovascular disorders and in monitoring various therapies for cardiovascular diseases.

15. The method of claim 1-2 to be used in the diagnosis of various cardiovascular disorders and in monitoring various therapies for cardiovascular diseases.

16. The method of claim 1 and 3 to be used in the diagnosis of various cardiovascular disorders and in monitoring various therapies for cardiovascular diseases.

17. The method of claim 1 and 3 to be used in the diagnosis of various cardiovascular disorders and in monitoring various therapies for cardiovascular diseases.

18. The method of claim 1 and 3 to be used in the diagnosis, of unstable angina, myocardial infarction and in monitoring various therapies for those diseases.

19. The method of claim 1 and 3 to be used in the diagnosis of stroke and in monitoring various therapies for stroke.

20. The method of claim 1 and 3 to be used in the diagnosis of various peripheral vascular disorders and in monitoring various therapies.

21. The method of claim 1 and 3 to be used in the diagnosis of various vascular disorders and in monitoring various therapies for vascular diseases.

22. The method of claim 1 and 3 to be used in the diagnosis of various acute/chronic inflammatory disorders and in monitoring various therapies for inflammatory diseases.

23. The method of claim 1 and 3 to be used in the diagnosis of various angiogenesis-mediated disorders and in monitoring various therapies for angiogenesis-mediated diseases.

24. The method of claim 1 wherein the biological sample is serum, plasma, urine, synovial fluid or spinal fluid.
